(19) United States
(12) Patent Application Publication
(10) Pub. No.: US 2011/0059979 A1
(43) Pub. Date: Mar. 10, 2011

(54) Piperidine/Cyclohexane Carboxamide Derivatives For Use as Vanilloid Receptor Modulators

(75) Inventors: Craig Jamieson, Lanarkshire (GB); David Drysdale Miller, Hertfordshire (GB); Harshad Kantilal Rami, Essex (GB); Mervyn Thompson, Essex (GB)

(73) Assignee: GLAXO GROUP LIMITED, Greenford Middlesex (GB)

(21) Appl. No.: 10/568,028

(22) PCT Filed: Aug. 12, 2004
(86) PCT No.: PCT/EP2004/009078

§ 371 Date: Mar. 25, 2008

(30) Foreign Application Priority Data
Aug. 14, 2003 (GB) 0319150.9

(51) Int. Cl. A61K 31/506; A61K 31/501; A61K 31/4709; A61K 31/4545; A61K 31/454; C07D 403/02; C07D 401/02

(52) U.S. Cl. 514/252.03; 514/275; 514/307; 514/314; 514/322; 514/318; 544/238; 544/331; 546/148; 546/176; 546/193; 546/198

(57) ABSTRACT

Certain compounds of formula (I),

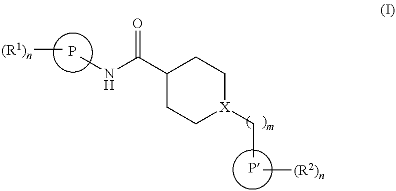

or a pharmaceutically acceptable salt or solvate thereof, wherein R¹, R², P, P′, X, m and n are as defined in the specification, a process for preparing such compounds, a pharmaceutical composition comprising such compounds and the use of such compounds in medicine.

This invention relates to novel amide derivatives having pharmacological activity, processes for their preparation, to compositions containing them and to their use in medicine, especially in the treatment of various disorders.

Vanilloids are a class of natural and synthetic compounds that are characterised by the presence of a vanillyl (4-hydroxy-3-methoxybenzyl) group or a functionally equivalent group. Vanilloid Receptor (VR-1), whose function is modulated by such compounds, has been widely studied and is extensively reviewed by Szallasi and Blumberg (The American Society for Pharmacology and Experimental Therapeutics, 1999, Vol. 51, No. 2.).

A wide variety of Vanilloid compounds of different structures are known in the art, for example those disclosed in European Patent Application Numbers, EP 0 347 000 and EP 0 401 903, UK Patent Application Number GB 2226313 and International Patent Applications, Publication Numbers WO 92/09285, WO 02/100819 and WO 02/076946. Particularly notable examples of vanilloid compounds or vanilloid receptor modulators are capsaicin or trans 8-methyl-N-vanillyl-6-nonenamide which is isolated from the pepper plant, capsazepine (Tetrahedron, 53, 1997, 4791) and olvanil or —N-(4-hydroxy-3-methoxybenzyl)oleamide (J. Med. Chem., 36, 1993, 2595).

International Patent Application, Publication Number WO 02/08221 discloses diaryl piperazine and related compounds which bind with high selectivity and high affinity to vanilloid receptors, especially Type I Vanilloid receptors, also known as capsaicin or VR1 receptors. The compounds are said to be useful in the treatment of chronic and acute pain conditions, itch and urinary incontinence.

International Patent Applications, Publication Numbers WO 02/16317, WO 02/16318 and WO 02/16319 suggest that compounds having a high affinity for the vanilloid receptor are useful for treating stomach-duodenal ulcers.

International Patent Applications, Publication Numbers WO 02/072536, WO 02/090326, WO 03/022809, WO 03/053945, WO 03/068749 and WO 04/024710; and co-pending International Patent Application Numbers PCT/GB2004/000543, PCT/EP2004/002377, PCT/GB2004/000978 and PCT/EP2004/002376 also describe a variety of compounds having activity as vanilloid receptor antagonists.

According to a first aspect of the present invention, there is provided a compound of formula (I),

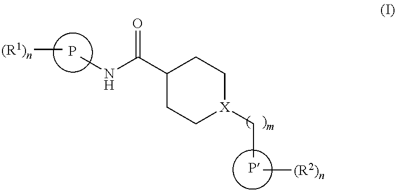

or a pharmaceutically acceptable salt or solvate thereof, wherein,
P represents phenyl, quinolinyl, isoquinolinyl, 1,2,3,4-tetrahydroquinolinyl, benzoisoxazolyl or benzothiazolyl;
P′ represents phenyl, pyridinyl, pyrimidinyl, pyridazinyl or benzothiazolyl;
R¹ and R² may be the same or different and represent alkyl, alkoxy, halo, —CF₃, —OCF₃, —OH, ═O, —CN, —NO₂, —SO₂NH₂, —SO₂R³ or —NR³R⁴;
R³ and R⁴ may be the same or different and represent —H or alkyl;
m represents 0 or 1;
n represents 0, 1, 2, 3, 4 or 5; and
X represents N or CH;
with the proviso that said compound of formula (I) is not a compound selected from:
4-Phenyl-N-quinolin-7-yl-piperidine-1-carboxamide;
N-Quinolin-7-yl-1-(5-trifluoromethylpyrid-2-yl)-piperidine-4-carboxamide;
N-Quinolin-7-yl-1-(6-trifluoromethylpyrid-2-yl)-piperidine-4-carboxamide;
N-Isoquinolin-5-yl-1-(5-trifluoromethylpyrid-2-yl)-piperidine-4-carboxamide; and
4-(4-Chlorophenyl)-N-(2-methylbenzothiazol-5-yl)cyclohexane-1-carboxamide.

Suitably, P represents phenyl, quinolinyl, isoquinolinyl, benzoisoxazolyl or benzothiazolyl. Preferably, P represents phenyl. Preferably, P represents quinolinyl, isoquinolinyl, benzoisoxazolyl or benzothiazolyl.

Preferably, P′ represents phenyl. Preferably, P′ represents pyridinyl or pyrimidinyl.

Preferably, R¹ represents alkyl such as methyl, halo such as chloro or bromo, ═O, —SO₂NH₂, or —SO₂Me.

Preferably, R² represents alkyl, alkoxy such as methoxy, halo such as chloro or fluoro, —CF₃ or —CN.

Preferably, R³ is —H or methyl.

Preferably, R⁴ is —H or methyl.

Preferably, m represents 0. Preferably, m represents 1.

Preferably, n represents 0, 1 or 2.

Preferably, X represents N. Preferably, X represents CH.

Preferred compounds according to this invention include Examples 1-49 or pharmaceutically acceptable salts or solvates thereof.

Particularly preferred compounds according to this invention include Examples 1, 3, 8, 16-25, 28-29, 31-33, 43-45.

Certain of the carbon atoms of formula (I) are chiral carbon atoms, and therefore compounds of formula (I) may exist as stereoisomers. The invention extends to all optical isomers such as stereoisomeric forms of the compounds of formula (I) including enantiomers and mixtures thereof, such as racemates. The different stereoisomeric forms may be separated or resolved one from the other by conventional methods or any given isomer may be obtained by conventional stereospecific or asymmetric syntheses.

As indicated above, the compounds of formula (I) can form salts, especially pharmaceutically acceptable salts. Suitable pharmaceutically acceptable salts are those used conventionally in the art and include those described in J. Pharm. Sci., 1977, 66, 1-19, such as acid addition salts.

Suitable pharmaceutically acceptable salts include acid addition salts.

Suitable pharmaceutically acceptable acid addition salts include salts with inorganic acids such, for example, as hydrochloric acid, hydrobromic acid, orthophosphoric acid or sulphuric acid, or with organic acids such, for example as methanesulphonic acid, toluenesulphonic acid, acetic acid, propionic acid, lactic acid, citric acid, fumaric acid, malic acid, succinic acid, salicylic acid, maleic acid, glycerophosphoric acid or acetylsalicylic acid.

The salts and/or solvates of the compounds of the formula (I) which are not pharmaceutically acceptable may be useful as intermediates in the preparation of pharmaceutically acceptable salts and/or solvates of compounds of formula (I) or the compounds of the formula (I) themselves, and as such form another aspect of the present invention.

The compounds of formula (I) may be prepared in crystalline or non-crystalline form, and if crystalline, may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

As used herein the term “alkyl” as a group or part of a group refers to a straight or branched chain saturated aliphatic hydrocarbon radical containing 1 to 12 carbon atoms, suitably 1 to 6 carbon atoms. Such alkyl groups in particular include methyl (“Me”), ethyl (“Et”), n-propyl (“Prⁿ”), iso-propyl (“Prⁱ”), n-butyl (“Buⁿ”), sec-butyl (“Bu^{s}”), tert-butyl (“Bu^{t}”), pentyl and hexyl. The term “cycloalkyl” as part of a group refers to a saturated alicyclic hydrocarbon radical containing 3 to 12 carbon atoms, suitably 3 to 6 carbon atoms. Where appropriate, such alkyl groups may be substituted by one or more groups selected from halo (such as fluoro, chloro, bromo), —CN, —CF₃, —OH, —OCF₃, C₂₋₆ alkenyl, C₃₋₆ alkynyl, C₁₋₆ alkoxy, aryl and di-C₁₋₆ alkylamino. Alkyl is preferably unsubstituted.

As used herein, the term “alkoxy” as a group or part of a group refers to an alkyl ether radical, wherein the term “alkyl” is defined above. Such alkoxy groups in particular include methoxy, ethoxy, n-propoxy, iso-propoxy, n-butoxy, iso-butoxy, sec-butoxy and tert-butoxy. Where appropriate, such alkoxy groups may be substituted by one or more groups selected from halo (such as fluoro, chloro, bromo), —CN, —CF₃, —OH, —OCF₃, C₁₋₆ alkyl, C₂₋₆ alkenyl, C₃₋₆ alkynyl, aryl and di-C₁₋₆ alkylamino. Alkoxy is preferably unsubstituted.

The term “halo” is used herein to describe, unless otherwise stated, a group selected from fluorine (“fluoro”), chlorine (“chloro”), bromine (“bromo”) or iodine (“iodo”).

The present invention also provides a process for the preparation of a compound of formula (I) or a pharmaceutically acceptable salt or solvate thereof, which process comprises:

(a) reacting a compound of formula (II),

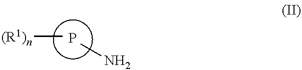

wherein, P, R¹ and n are as defined in relation to formula (I), with a compound of formula (III),

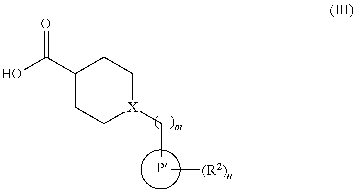

wherein, P′, R², m, n and X are as defined in relation to formula (I) and thereafter, as necessary, carrying out one or more of the following reactions:
(i) converting one compound of formula (I) into another compound of formula (I);
(ii) removing any protecting group;
(iii) preparing a salt or a solvate of the compound so formed.

The reaction between a compound of formula (II) and a compound of formula (III) may be effected using conventional methods for the formation of an amide bond, such as those described in J March, Advanced Organic Chemistry, 4th edition, J Wiley & Sons, 1992, p. 419-421.

Compounds of formula (II) are either commercially available, or may be prepared by the reaction of a compound of formula (IV),

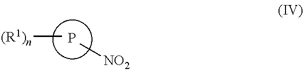

wherein, P, R¹ and n are as defined in relation to formula (I), with a suitable reducing agent.

The reaction of a compound of formula (IV) with a reducing agent may be effected by methods well known in the art, such as those described in J March, Advanced Organic Chemistry, 4th edition, J Wiley & Sons, 1992, p. 1216-1218. Suitable reducing agents include (a) iron or zinc metal in hydrochloric acid, or (b) hydrogen in the presence of a suitable catalyst, such as, 5% palladium on charcoal. Reduction using hydrogen may conveniently be performed in a solvent such as methanol or ethanol.

Compounds of formula (IV) are commercially available or may be prepared according to literature methods, such as those described in Larock R. F. ‘Comprehensive Organic Transformations”, New York, Wiley (1999), under conditions determined by the particular groups chosen.

Compounds of formula (III) are either commercially available or may be prepared by hydrolysis of a compound of formula (V),

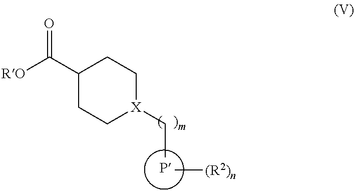

wherein, P′, R², m, n and X are as defined in relation to formula (I) and R′ is an alkyl group. A suitable hydrolysis agent is aqueous hydrochloric acid. A suitable solvent is dioxane.

Compounds of formula (V) are commercially available or may be prepared according to literature methods such as those described in J. Org. Chem. 28, 1963, 3259 or J. Am. Chem. Soc. 118, 1996, 7215.

The above-mentioned conversions of a compound of formula (I) into another compound of formula (I) include any conversion, which may be effected using conventional procedures, but in particular the said conversions include any combination of:

(i) converting one group R¹ into another group R¹; and
(ii) converting one group R² into another group R².

The above-mentioned conversions (i)-(ii) may be performed using any appropriate method under conditions determined by the particular groups chosen.

It will be appreciated by those skilled in the art that it may be necessary to protect certain reactive substituents during some of the above procedures. Standard protection and deprotection techniques, such as those described in Greene T. W. ‘ Protective groups in organic synthesis’, New York, Wiley (1981), can be used. For example, primary amines can be protected as phthalimide, benzyl, benzyloxycarbonyl or trityl derivatives. Carboxylic acid groups can be protected as esters. Aldehyde or ketone groups can be protected as acetals, ketals, thioacetals or thioketals. Deprotection of such groups is achieved using conventional procedures known in the art.

Pharmaceutically acceptable salts may be prepared conventionally by reaction with the appropriate acid or acid derivative.

Compounds of formula (I) and their pharmaceutically acceptable salts and solvates thereof have Vanilloid receptor antagonist (VR1) activity and are believed to be of potential use for the treatment or prophylaxis of certain disorders, or treatment of the pain associated with them, such as: pain, chronic pain, neuropathic pain, postoperative pain, postrheumatoid arthritic pain, osteoarthritic pain, back pain, visceral pain, cancer pain, algesia, neuralgia, dental pain, headache, migraine, neuropathies, carpal tunnel syndrome, diabetic neuropathy, HIV-related neuropathy, post-herpetic neuralgia, fibromyalgia, neuritis, sciatica, nerve injury, ischaemia, neurodegeneration, stroke, post stroke pain, multiple sclerosis, respiratory diseases, asthma, cough, COPD, broncho constriction, inflammatory disorders, oesophagitis, heart burn, Barrett's metaplasia, dysphagia, gastroeosophageal relux disorder (GERD), stomach and duodenal ulcers, functional dyspepsia, irritable bowel syndrome, inflammatory bowel disease, colitis, Crohn's disease, pelvic hypersensitivity, pelvic pain, menstrual pain, renal colic, urinary incontinence, cystitis, burns, itch, psoriasis, pruritis, emesis (hereinafter referred to as the “Disorders of the Invention”).

Accordingly, the invention also provides a compound of formula (I) or a pharmaceutically acceptable salt or solvate thereof, for use as an active therapeutic substance, in particular, in the treatment and/or prophylaxis of the Disorders of the Invention.

In particular, the invention provides a compound of formula (I) or a pharmaceutically acceptable salt or solvate thereof for use in the treatment or prophylaxis of pain.

The invention further provides a method for the treatment or prophylaxis of disorders in which antagonism of the Vanilloid (VR1) receptor is beneficial, in particular the Disorders of the Invention, in mammals including humans, which method comprises administering to a mammal in need thereof a therapeutically effective amount of a compound of formula (I) or a pharmaceutically acceptable salt or solvate thereof.

The invention provides for the use of a compound of formula (I) or a pharmaceutically acceptable salt or solvate thereof in the manufacture of a medicament for the treatment or prophylaxis of disorders in which antagonism of the Vanilloid (VR1) receptor is beneficial, particularly the Disorders of the Invention.

In order to use the compounds of the invention in therapy, they will normally be formulated into a pharmaceutical composition in accordance with standard pharmaceutical practice. Thus, the present invention also provides a pharmaceutical composition, which comprises a compound of formula (I) or a pharmaceutically acceptable salt or solvate thereof and a pharmaceutically acceptable carrier or excipient therefor.

A pharmaceutical composition of the invention, which may be prepared by admixture, suitably at ambient temperature and atmospheric pressure, is usually adapted for oral, parenteral, rectal administration or intravesical administration to the bladder and, as such, may be in the form of tablets, capsules, oral liquid preparations, powders, granules, lozenges, reconstitutable powders, injectable or infusable solutions, suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. For systemic administration, dosage levels from 0.01 mg to 100 mg per kilogram of body weight are useful in the treatment of pain. However, as a general guide suitable unit doses may be 0.05 to 1000 mg, more suitably 0.05 to 20, 20 to 250, or 0.1 to 500.0 mg, for example 0.2 to 5 and 0.1 to 250 mg; and such unit doses may be administered more than once a day, for example two or three a day, so that the total daily dosage is in the range of about 0.5 to 1000 mg; and such therapy may extend for a number of weeks or months.

No unacceptable toxicological effects are indicated with compounds of the invention when administered in accordance with the invention.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of the compounds of the invention.

### Abbreviations

DMF=dimethylformamide, DCM=dichloromethane,
BINAP=2,2′-bis(diphenylphosphino)-1,1′-binaphthyl,
NaOH=sodium hydroxide, LiOH=lithium hydroxide

### Description 1

### 5-Nitro-1-methylquinolinium iodide (D1)

A mixture of 5-nitroquinoline (5 g, 0.028 mol) and iodomethane (5.4 ml, 0.086 mol) in DMF (8 ml) was heated to 40° C. After 2 h a thick dark red precipitate formed, the mixture was cooled and diluted with acetone. The solid was filtered, washed with acetone and dried to give the title compound as an orange solid.

### Description 2

### 5-Nitro-1-methyl-2-(1H)-quinolinone (D2)

A solution of D1 (8.47 g, 0.03 mol) in warm water (90 ml) was added dropwise to a solution of potassium ferricyanide (33.6 g, 0.1 mol) in 10% NaOH maintained at 45° C. After 5 h the temperature was raised to 60° C. and the solution heated for ca. 24 h. The solution was cooled in an ice bath for 15 min and the grey-green solid filtered off, washed with water and dried. The crude solid was dissolved in a minimum amount of DCM, filtered through silica gel and washed with ethyl acetate until no further product was eluted. Evaporation in vacuo afforded the title compound as a dark orange solid.

### Description 3

### 5-Amino-1-methyl-2-(1H)-quinolinone (D3)

D2 (2.59 g, 0.13 mol) in ethanol (100 ml) and DMF (30 ml) was treated with 10% palladium on charcoal, (1 g, 50% w/w water). The mixture was hydrogenated for a 8 h at atmospheric pressure and, after removal of the catalyst, concentrated in vacuo. Trituration with ether afforded the title compound as a buff solid.

### Description 4

### 1-(4-Chlorophenyl)piperidine-4-carboxylic acid, ethyl ester (D4)

Racemic BINAP (2.25 g, 0.0036 mol), palladium acetate (0.82 g, 3.65 mmol) and caesium carbonate (16.86 g, 0.051 mol) were suspended in 1,4-dioxane (100 ml) and sonicated for 45 min. 4-Bromo-chlorobenzene (5 g, 26.12 mmol) and ethyl isonipecoacetate (4.11 g, 26.12 mmol) were added as a solution in 1,4-dioxane (100 ml). The mixture was heated to 105° C. for 16 h. On cooling the solvent was removed and the residues partitioned between water (100 ml) and diethyl ether (100 ml); the aqueous was re-extracted with ether. The combined layers were dried (Na₂SO₄) and concentrated in vacuo. Purification by flash chromatography (ethylacetate/pet. ether) gave the title compound as an oil.

### Description 5

### 1-(4-Chloro-phenyl)-piperidine-4-carboxylic acid (D5)

A solution of D4 (1.82 g, 0.0067 mol) in 1N LiOH (30 ml) and dioxane (30 ml) was stirred at room temperature for 16 h and then evaporated in vacuo. Work-up with 1M HCl and ethyl acetate gave the title compound as a yellow solid.

The following were prepared using a method similar to that employed in Descriptions 4 and 5.
1-(3-Chlorophenyl)-piperidine-4-carboxylic acid (D6)
1-(4-Cyanophenyl)-piperidine-4-carboxylic acid (D7)
1-(4-Fluorophenyl)-piperidine-4-carboxylic acid (D8)
1-(4-Methylphenyl)-piperidine-4-carboxylic acid (D9)
1-(4-Methoxyphenyl)-piperidine-4-carboxylic acid (D10)
1-(4-Trifluoromethylphenyl)-piperidine-4-carboxylic acid (D11)
1-(4-Chloro-2-methylphenyl)-piperidine-4-carboxylic acid (D12)
1-(4-Chloro-2-trifluoromethylphenyl)-piperidine-4-carboxylic acid (D13)
1-(2,4-Dichlorophenyl)-piperidine-4-carboxylic acid (D14)
1-(2,5-Dichlorophenyl)-piperidine-4-carboxylic acid (D15)
1-(3,5-Dihlorophenyl)-piperidine-4-carboxylic acid (D16)
1-(3,4-Dichloro-phenyl)-piperidine-4-carboxylic acid (D17)
1-(2-Chloro-4-fluorophenyl)-piperidine-4-carboxylic acid (D18)
1-(4-Chloro-3-fluorophenyl)-piperidine-4-carboxylic acid (D19)
1-(2,4-Difluorophenyl)-piperidine-4-carboxylic acid (D20)
1-(5-Chloropyridin-2-yl)-piperidine-4-carboxylic acid (D21)
1-(5-Trifluoromethylpyridin-2-yl)-piperidine-4-carboxylic acid (D22)
1-(3-Chloro-5-trifluoromethylpyridin-2-yl)-piperidine-4-carboxylic acid (D23)
1-(3-Trifluoromethylpyridin-2-yl)-piperidine-4-carboxylic acid (D24)
1-(6-Methyl-4-trifluoromethylpyridin-2-yl)-piperidine-4-carboxylic acid (D25)
1-(Pyrimidin-2-yl)-piperidine-4-carboxylic acid (D26)
1-(4-Trifluoromethylpyrimidin-2-yl)-piperidine-4-carboxylic acid (D27)
1-(6 Chloropyridazin-3-yl)-piperidine-4-carboxylic acid (D28)
1-(Benzothiazol-2-yl)-piperidine-4-carboxylic acid (D29)

The following were prepared using a method similar to that employed in F. E. Blaney et al., J. Med. Chem., 1983, 26, 1747.
1-(Benzyl)-piperidine-4-carboxylic acid (D30)
1-(2-Chlorobenzyl)-piperidine-4-carboxylic acid (D31)
1-(3-Chlorobenzyl)-piperidine-4-carboxylic acid (D32)
1-(4-Chlorobenzyl)-piperidine-4-carboxylic acid (D33)
1-(2,4-Dichlorobenzyl)-piperidine-4-carboxylic acid (D34)

### Description 35

### 6-Aminobenzisoxazole (D35)

The title compound was prepared from 6-nitrobenzoisoxazole (F. Hollfelder et al., J. Org. Chem., 2001, 66, 5866) by reduction using methods in WO 2004/024710.
5-Amino-2-methylbenzothiazole, 5-aminoisoquinoline and 5-aminoquinoline are commercially available. 5-Amino-1-methylisoquinoline was prepared according to WO 2004/024710.

### EXAMPLE 1

### 1-(4-Chlorophenyl)-N-(1-methyl-2-oxo-1,2-dihydro-5-quinolinyl)-4-piperidinecarboxamide (E1)

A suspension of 1-(4-chlorophenyl)-4-piperidinecarboxylic acid, (D5, 150 mg, 0.63 mM) in DCM (5 ml) under argon, was treated with oxalyl chloride, (0.164 ml, 1.88 mM) and 1 drop of DMF. After 2 h, the solution was concentrated in vacuo and then redissolved in DCM (10 ml). The solution was cooled in an ice-bath and treated with a solution of 5-amino-1-methyl-2-(1H)-quinolinone, D3 (109 mg, 0.03 mmol) and pyridine, (0.061 ml, 0.75 mmol). The mixture was kept at 25° C. for ca. 2 h, then 45° C. for a further 2 h and 24 h at 25° C. again. The thick precipitate formed was removed by centrifugation washed with DCM, ether and dried to give the title compound as a buff solid, (156 mg, 63%). MH⁺ 396, 394.

### EXAMPLE 2

### 4-(4-Chlorophenyl)-N-5-quinolinylcyclohexanecarboxamide (E2)

To a solution of 4-(4-chlorophenyl)cyclohexanecarboxylic acid (11.9 mg, 0.05 mmol) in N,N-dimethylacetamide (1 mL) was added thionyl chloride (2.0 M solution in DCM (25 μL, 0.05 mmol) and the resultant solution stirred for 30 min. 5-Aminoquinoline (7.2 mg, 0.05 mmol) and diisopropylethylamine (19 μL, 0.15 mmol) in N,N-dimethylacetamide (0.5 mL) were added. The mixture stirred for 16 h and then evaporated in vacuo. Purification of the residue by reverse phase HPLC gave the title compound as a white solid (6.0 mg, 33%). MH⁺=365, 363.
Examples 3-49 presented in Table 1 were prepared by procedures similar to those described in Examples 1 and 2.

TABLE 1

| Example No | Structure | MH+ (observed) |
|---|---|---|
| 3 | 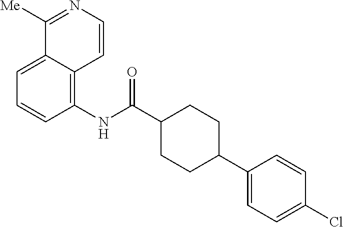 | 379, 377 |
| 4 | 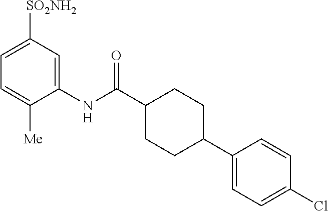 | 407, 405 |
| 5 | 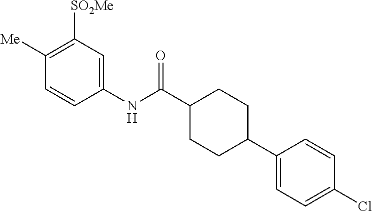 | 406, 404 |
| 6 | 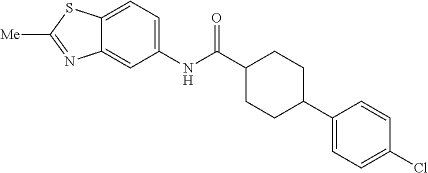 | 385, 383 |
| 7 | 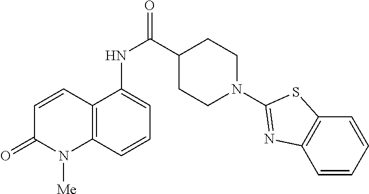 | 419 |
| 8 | 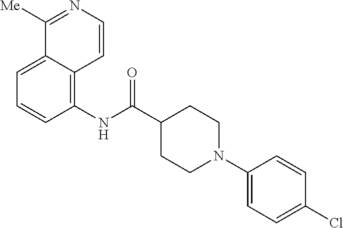 | 380, 378 |
| 9 | 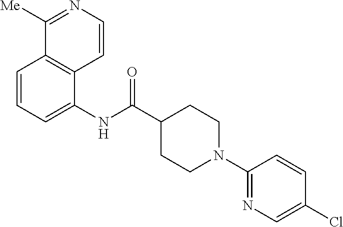 | 381, 379 |
| 10 | 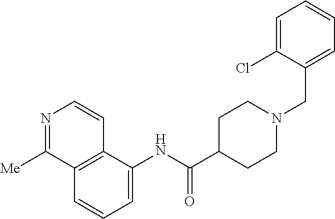 | 394, 392 |
| 11 | 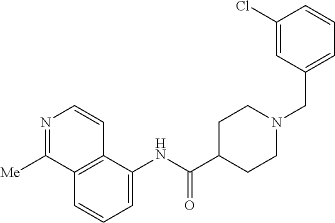 | 394, 392 |
| 12 | 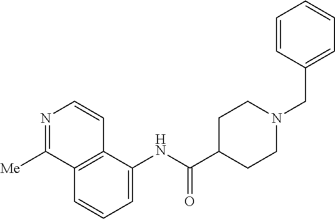 | 360 |
| 13 | 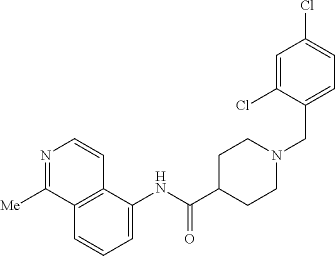 | 430, 428 |
| 14 | 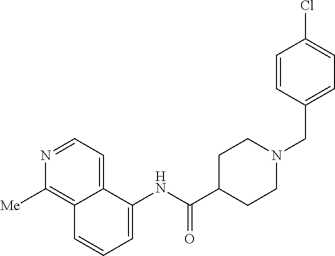 | 394, 392 |
| 15 | 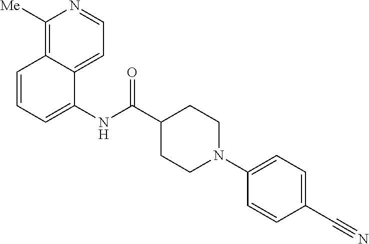 | 371 |
| 16 | 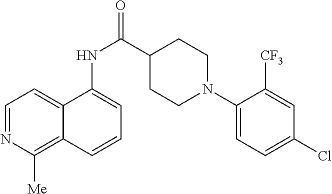 | 448, 446 |
| 17 | 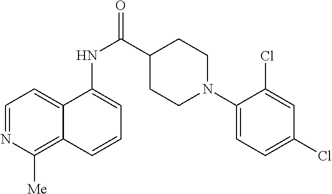 | 416, 414 |
| 18 | 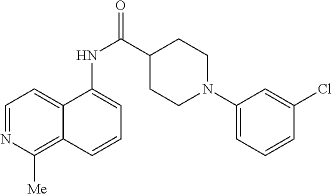 | 380, 378 |
| 19 | 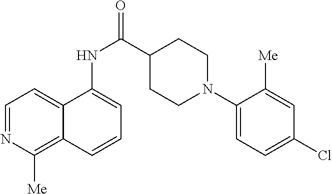 | 394, 392 |
| 20 | 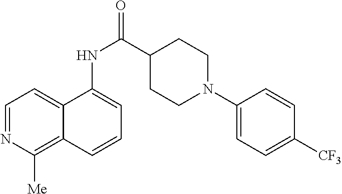 | 414 |
| 21 | 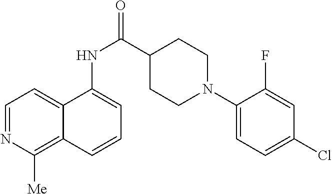 | 398, 396 |
| 22 | 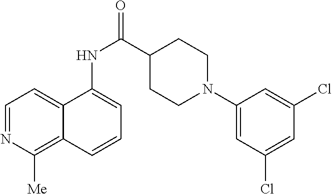 | 414 |
| 23 | 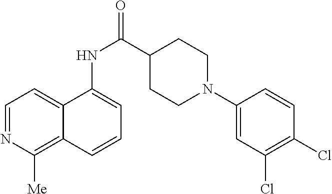 | 416, 414 |
| 24 | 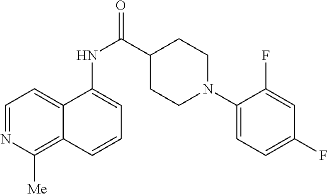 | 382 |
| 25 | 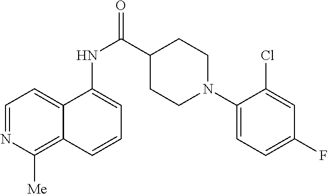 | 398, 396 |
| 26 | 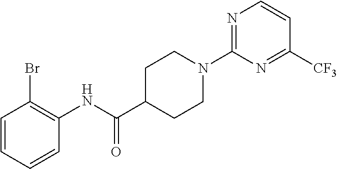 | 431, 429 |
| 27 | 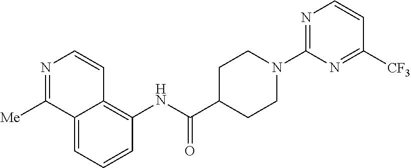 | 416 |
| 28 | 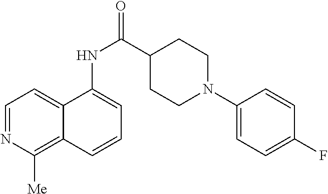 | 364 |
| 29 | 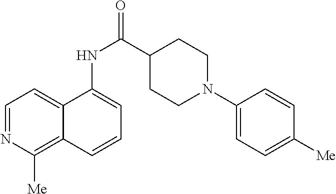 | 360 |
| 30 | 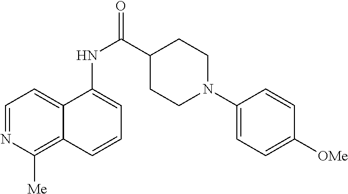 | 376 |
| 31 | 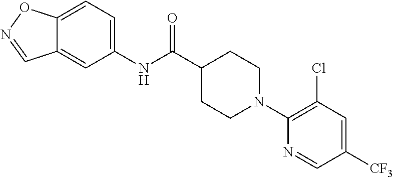 | 425, 423 |
| 32 | 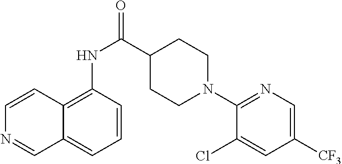 | 435, 433 |
| 33 | 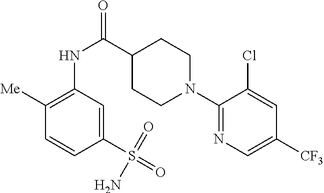 | 477, 475 |
| 34 | 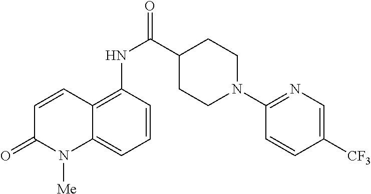 | 431 |
| 35 | 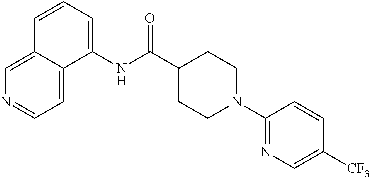 | 401 |
| 36 | 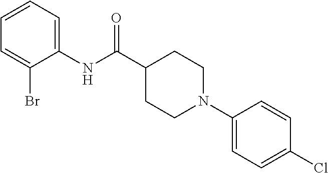 | 394 |
| 37 | 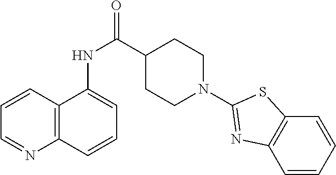 | 389 |
| 38 | 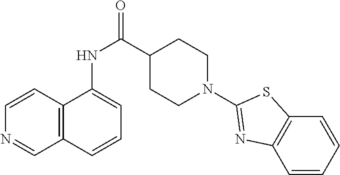 | 389 |
| 39 | 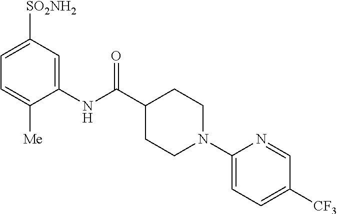 | 443 |
| 40 | 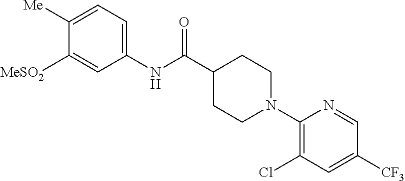 | 476, 474 |
| 41 | 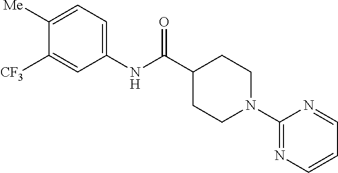 | 365 |
| 42 | 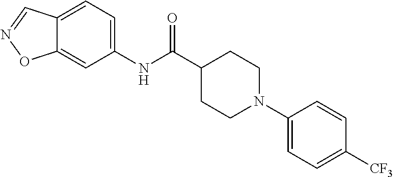 | 391 |
| 43 | 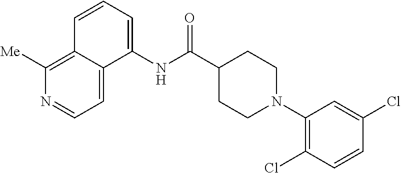 | 416, 414 |
| 44 | 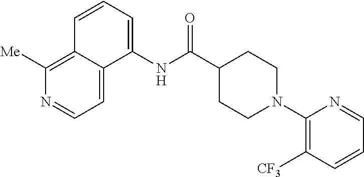 | 415 |
| 45 | 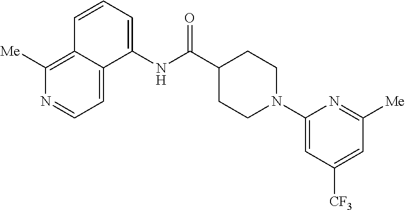 | 429 |
| 46 | 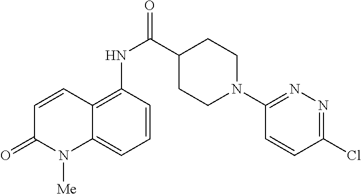 | 398, 396 |
| 47 | 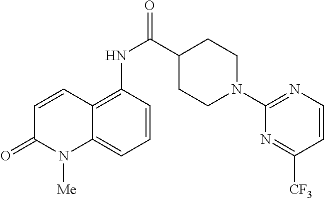 | 432 |
| 48 | 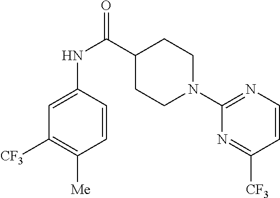 | 433 |
| 49 | 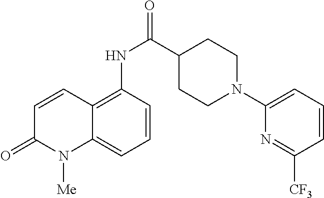 | 431 |

### Pharmacological Data

### (a) In Vitro Assay

As referenced above, the compounds of the invention are vanilloid receptor (VR1) antagonists and hence have useful pharmaceutical properties. Vanilloid receptor (VR1) antagonist activity can be confirmed and demonstrated for any particular compound by use of conventional methods, for example those disclosed in standard reference texts such as D. Le Bars, M. Gozarin and S. W. Cadden, Pharmacological Reviews, 2001, 53(4), 597-652] or such other texts mentioned herein.

The screen used for the compounds of this invention was based upon a FLIPR based calcium assay, similar to that described by Smart et al. (British Journal of Pharmacology, 2000, 129, 227-230).

Transfected astrocytoma 1321N1 cells, stably expressing human VR1, were seeded into FLIPR plates at 25,000 cells/well (96-well plate) and cultured overnight.

The cells were subsequently loaded in medium containing 4 μM Fluo-3 AM (Molecular Probes) for 2 hours, at room temperature, in the dark. The plates were then washed 4 times with Tyrode containing 1.5 mM calcium, without probenecid.

The cells were pre-incubated with compound or buffer control at room temperature for 30 minutes. Capsaicin (Sigma) was then added to the cells. Compounds having antagonist activity against the human VR1 were identified by detecting differences in fluorescence when measured after capsaicin addition, compared with no compound buffer controls. Thus, for example, in the buffer control capsaicin addition results in an increase in intracellular calcium concentration resulting in fluorescence. A compound having antagonist activity blocks the capsaicin binding to the receptor, there is no signalling and therefore no increase in intracellular calcium levels and consequently lower fluorescence. pKb values are generated from the IC₅₀ values using the Cheng-Prusoff equation.

All compounds tested by the above methodology had pKb>5, preferred compounds (Examples 1, 3, 8, 16-25, 28-29, 31-33, 43-45) having a pKb>7.0.

### (b) FCA-Induced Hyperalgesia in the Guinea Pig

Compounds having a pKb>7.0 in vitro, according to model (a) were tested in this model of hyperalgesia (see WO 2004/024710 for details) and shown to be active. Example 1 had significant activity at a dose of 5 mg/kg po.

What is claimed is:

1. A compound of formula (I), 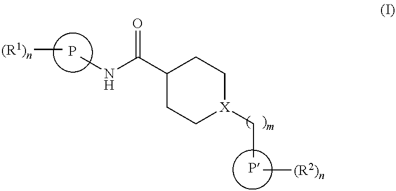
or a pharmaceutically acceptable salt or solvate thereof, wherein,
P represents phenyl, quinolinyl, isoquinolinyl, 1,2,3,4-tetrahydroquinolinyl, benzoisoxazolyl or benzothiazolyl;
P′ represents phenyl, pyridinyl, pyrimidinyl, pyridazinyl or benzothiazolyl;
R¹ and R² may be the same or different and represent alkyl, alkoxy, halo, —CF₃, —OCF₃, —OH, ═O, —CN, —NO₂, —SO₂NH₂, —SO₂R³ or —NR³R⁴;
R³ and R⁴ may be the same or different and represent —H or alkyl;
m represents 0 or 1;
n represents 0, 1, 2, 3, 4 or 5; and
X represents N or CH;
with the proviso that said compound of formula (I) is not a compound selected from:
4-Phenyl-N-quinolin-7-yl-piperidine-1-carboxamide;
N-Quinolin-7-yl-1-(5-trifluoromethylpyrid-2-yl)-piperidine-4-carboxamide;
N-Quinolin-7-yl-1-(6-trifluoromethylpyrid-2-yl)-piperidine-4-carboxamide;
N-Isoquinolin-5-yl-1-(5-trifluoromethylpyrid-2-yl)-piperidine-4-carboxamide; and
4-(4-Chlorophenyl)-N-(2-methylbenzothiazol-5-yl)cyclohexane-1-carboxamide.

2. A compound of formula (I), as claimed in claim 1, wherein P represents phenyl, quinolinyl, isoquinolinyl, benzoisoxazolyl or benzothiazolyl.

3. A compound of formula (I), as claimed in claim 2, wherein P represents phenyl.

4. A compound of formula (I), as claimed in claim 2, wherein P represents quinolinyl, isoquinolinyl, benzoisoxazolyl or benzothiazolyl.

5. A compound of formula (I), as claimed in claim 1, wherein P′ represents phenyl.

6. A compound of formula (I), as claimed in claim 1, wherein P′ represents pyridinyl or pyrimidinyl.

7. A compound of formula (I) or a pharmaceutically acceptable salt or solvate thereof, as claimed in claim 1, substantially as hereinbefore described with reference to any one of the Examples.

8. A process for the preparation of a compound of formula (I) or a pharmaceutically acceptable salt or solvate thereof, as claimed in claim 1, which process comprises:
(a) reacting a compound of formula (II), 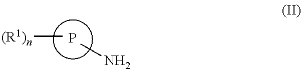
wherein, P, R¹ and n are as defined in relation to formula (I), with a compound of formula (III), 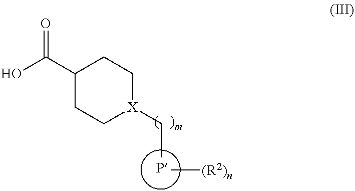
wherein, P′, R², m, n and X are as defined in relation to formula (I) and thereafter, as necessary, carrying out one or more of the following reactions:
(i) converting one compound of formula (I) into another compound of formula (I);
(ii) removing any protecting group;
(iii) preparing a salt or a solvate of the compound so formed.

9. A pharmaceutical composition, which comprises a compound of formula (I) or a pharmaceutically acceptable salt or solvate thereof, as claimed in claim 1, and a pharmaceutically acceptable carrier or excipient therefor.

10. (canceled)

11. A method for the treatment or prophylaxis of disorders in which antagonism of the Vanilloid (VR1) receptor is beneficial, in particular the Disorders of the Invention, in mammals including humans, which method comprises administering to a mammal in need thereof a therapeutically effective amount of a compound of formula (I) or a pharmaceutically acceptable salt or solvate thereof, as claimed in claim 1.

12. (canceled)
